(19) United States
(12) Patent Application Publication
(10) Pub. No.: US 2010/0104602 A1
(43) Pub. Date: Apr. 29, 2010

(54) Neurotoxin therapy for postprandial hyperglycemia

(76) Inventor: Pankaj J. Pasricha, Cupertino, CA (US)

Correspondence Address: Dr. Benjamin Adler;ADLER & ASSOCIATES, 8011 Candle Lane, Houston, TX, 77071, US

(21) Appl. No.: 12/655,214

(22) Filed: Dec. 24, 2009

(30) Foreign Application Priority Data
Jun. 25, 2008 (US) PCT/US2008/007659

(51) Int. Cl. A61K 39/08; A61P 3/10

(52) U.S. Cl. 424/239.1

(57) ABSTRACT

Botulinum toxin is increasingly being injected into visceral smooth muscle for a variety of indications. The present invention discloses intragastric administration of botulinum toxin to delay gastric emptying with the aim of inducing satiety and promoting weight-loss. The present invention also discloses the effects of intragastric administration of Botulinum toxin in reducing post-prandial hyperglycemia in patients suffering from Diabetes Mellitus.

### CROSS-REFERENCE TO RELATED APPLICATIONS

This application claims benefit of priority under 35 U.S.C. §120 of international application PCT/US2008/007659, filed Jun. 25, 2008, which claims benefit of priority under 35 U.S.C. §119(e) of provisional U.S. Serial No. 60/937,109, filed Jun. 25, 2007, now abandoned, the entirety of both of which are hereby incorporated by reference.

### BACKGROUND OF THE INVENTION

1. Field of the Invention

The present invention relates generally to the field of gastroenterology. Specifically the present invention discloses intragastric administration of botulinum toxin for the treatment and reduction of post-prandial hyperglycemia.

2. Description of the Related Art

Although the use of Botulinum neurotoxin (BoNT) for obesity was speculated about more than a decade ago, it is only recently that this approach has begun to be tested clinically. The rationale for its use is fairly simplistic. If Botulinum neurotoxin injection can impair gastric muscle contractions, then gastric emptying may be slowed, leading to increased retention of a meal with consequent satiety and weight loss. Gui et al were the first to demonstrate that intragastric injection of Botulinum neurotoxin could diminish food intake and prevent the normal increase in weight that control rats experienced. A further study by Coskun et al in obese rats confirmed this effect and also demonstrated an associated significant reduction of gastric emptying.

In 2003, the first case of weight loss in an obese patient was reported (Rollnik et al) after antral injection of a 100 units of botulinum neurotoxin type A. Four months after injection this patient had a 32.5% reduction in daily calorie intake and a 9% weight loss. Subsequently, small observational studies were inconclusive probably because of small sample size. Most recently, Foschi et al blindly randomized 24 morbidly obese patients (mean BMI of 43.67 kg/m2) to receive 200 IU botulinum neurotoxin type A or placebo into the antrum and fundus of the stomach endoscopically. Eight weeks after treatment, botulinum neurotoxin type A patients had significantly higher weight loss (11.7 vs 5.7 kg, P<0.001) and BMI reduction (4 vs 2 kg/m2, P<0.001) and a higher satiety score on a visual analog scale (7.63 vs 4.72, P<0.001) than controls. Furthermore, botulinum neurotoxin treated patients showed a significantly greater reduction in maximal gastric capacity for liquids (266 vs 139 ml, P<0.001) and a greater prolongation in gastric emptying time. No significant side effects or neurophysiologic changes were found.

As gastric emptying is a major determinant of postprandial hyperglycemia (PPGH), intragastric botulinum neurotoxin injection may have an effect on post-prandial glycemic control (PPHG) in diabetes mellitus. Postprandial hyperglycemia has been shown to increase the risk of complications in patients with diabetes mellitus. This has led to growing interest in treatments that specifically prevent post-prandial glycemic control. These drugs include alpha-Glucosidase inhibitors like acarbose and miglitol, which slow absorption of sucrose. The nonsulfonylurea secretagogues repaglinide and nateglinide provoke rapid secretion of endogenous insulin with meals.

These agents may reduce but do not eliminate post-prandial glycemic control. The use of rapid-acting analogs of insulin also generally does not result in complete prevention of postprandial hyperglycemia. Residual postprandial hyperglycemia contributes to the abnormal glycemic exposure of tissues and limits efforts to reduce A1C from ˜7% to the normal 4-6% range. Reducing postprandial hyperglycemia by more than 50% (which is probably all that these agents can do) will therefore probably require methods of treatment that are more effective than those currently available. Newly approved agents that may fulfill this promise include the amylin analog pramilintide and exanatide the GLP-1 agonist. Pramlintide acts principally by slowing gastric emptying (thought to be a central effect) and reducing flux of nutrients into the intestine. In addition, it suppresses plasma levels of glucagon, increases satiety, and blunts postprandial hyperglycemia dramatically. Thus a rationale for using botulinum toxin over and above its effect on weight loss becomes apparent. If it is effective in inhibiting gastric emptying, then as with pramlintide, it should significantly attenuate post-prandial hyperglycemia

Despite the overwhelming evidence regarding the potential effects of botulinum neurotoxin in weight loss and its possible use for reducing postprandial hyperglycemia, many important questions remain to be resolved. For one, the prior art does not teach the mechanism of action of botulinum neurotoxin in the treatment of obesity. The proposed mechanisms include either decreased gastric emptying or alteration in neuropeptide release (e.g. Ghrelin) or both. Ghrelin is one of the most important orexigenic (hunger promoting) hormones and is produced by specialized GRL cells mainly found lining the proximal part of the stomach. Decreases in ghrelin are therefore expected to reduce appetite; indeed low levels have been noted after gastric bypass surgery and may represent a more likely mechanism of weight loss than either gastric reduction or malabsorption. This may particularly account for why fundic injections of botulinum neurotoxin may result in weight loss. An alternative explanation of course is that fundal injection results in relaxation of the proximal stomach with greater storage capacity and decreased distal transit. Secondly, prior art also does not teach the optimal site of injection (Fundus, antrum or both), dose or the use of other serotypes of botulinum neurotoxin including Botulinum neurotoxin. Further, there exist numerous serotypes of the botulinum toxin.

The prior art does not teach the effects of Botulinum neurotoxin, in particular, in promoting weight loss and post-prandial fluctuations in glucose and hormonal levels. The present invention full fills this long-standing need in the art.

### SUMMARY OF THE INVENTION

The present invention is directed to a method for treating obesity in an individual consisting of intragastric administration of a neurotoxin to impair gastric muscle contraction, thereby effectively treating obesity in the individual. The present invention is also directed to a method for treatment of post-prandial hyperglycemia in an individual consisting of intragastric administration of a neurotoxin to impair gastric muscle contraction, thereby effectively treating post-prandial hyperglycemia in the individual.

The present invention is further directed to a method of inducing flaccid paralysis of the gastric muscle in an individual consisting of intragastric administration of Botulinum toxin B, where the method delays gastric emptying in the individual.

Other and further aspects, features and advantages of the present invention will be apparent from the following description of the embodiments of the invention given for the purpose of the disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows plasma glucose increments after a meal in 19 subjects with a meal in 19 subjects with type 1 diabetes taking regular insulin alone (dashed line) or with 60 mg Pramlintide (solid line) before the meal.

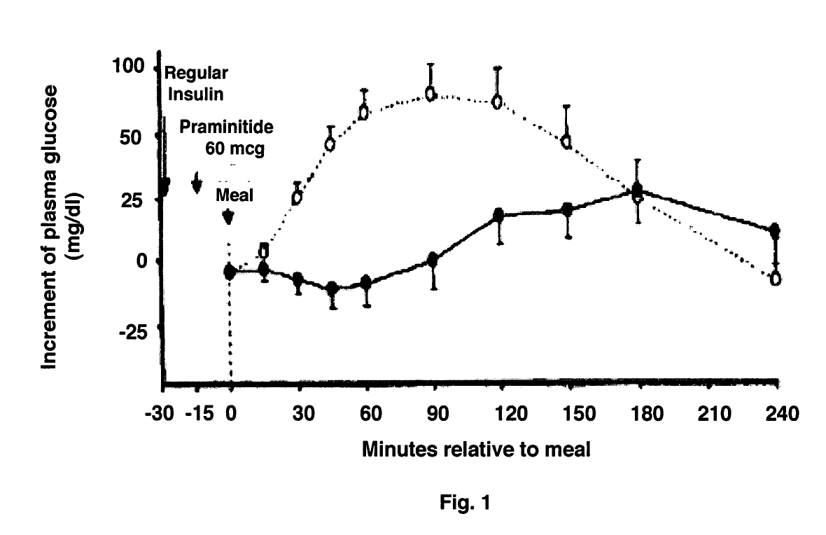

### DETAILED DESCRIPTION OF THE INVENTION

In one embodiment of the present invention, there is provided a method of treating obesity in an individual consisting of intragastric administration of a neurotoxin to impair gastric muscle contraction, thereby effectively treating obesity in the individual. The neurotoxin is administered in doses ranging from 1,000 units/Kg to 30,000 units/Kg of body weight. In general, the neurotoxin is made by a bacterium selected from the group consisting of Clostridium botulinum, Clostridium butyricum, and Clostridium beratti. More specifically, the neurotoxin is a botulinum toxin. Moreover, the botulinum toxin may be selected from the group consisting of botulinum toxin types A, B, C1, D, E, F and G.

In one embodiment of the present invention the neurotoxin is administered into the antrum of the stomach. In another embodiment, of the present invention, the neurotoxin is administered into the fundus of the stomach. In a related embodiment the neurotoxin is administered into both the antrum and the fundus of the stomach. In general, the impairment of the gastric muscle contraction results in a delayed emptying of the stomach. Additionally, the impairment of the gastric muscle contraction results in altered neuropeptide release. Moreover, the delay in emptying of the stomach promotes a sense of satiety. Specifically, the neuropeptide is gherlin.

In another embodiment of the present invention there is disclosed a method for treatment of post-prandial hyperglycemia in an individual consisting of intragastric administration of a neurotoxin to impair gastric muscle contraction, thereby effectively treating post-prandial hyperglycemia in the individual. Specifically, the neurotoxin is administered in doses ranging from 1,000 units/Kg to 30,000 units/Kg of body weight. The neurotoxin is made by a bacterium selected from the group consisting of Clostridium botulinum, Clostridium butyricum, and Clostridium baratti. Further, the neurotoxin is a botulinum toxin. In general, the botulinum toxin may be selected from the group consisting of botulinum toxin types A, B, C1, D, E, F and G. In one embodiment of the present invention the neurotoxin is administered into the antrum of the stomach. In another embodiment of the present invention the neurotoxin is administered into the fundus of the stomach.

In a related embodiment, of the present invention, the neurotoxin is administered into both the antrum and the fundus of the stomach. Specifically, the impairment of the gastric muscle contraction results in a delayed emptying of the stomach. Additionally, the impairment of the gastric muscle contraction results in altered neuropeptide release. The delay in emptying of the stomach promotes a sense of satiety. Specifically, the neuropeptide is gherlin. In general, the individual has overt diabetes mellitus.

In yet another embodiment of the present invention, there is provided a method of inducing flaccid paralysis of the gastric muscle in an individual consisting of intragastric administration of Botulinum toxin B, where the method delays gastric emptying in the individual. Specifically, the individual is suffering from Diabetes Mellitus. Moreover, the individual suffers from obesity. In one aspect of the present invention the toxin is administered into the antrum of the stomach. In another aspect of the present invention the toxin is administered into the fundus of the stomach. In a related aspect of the present invention the toxin is administered into both the antrum and the fundus of the stomach. In general, the delayed gastric emptying results in reduction of postprandial hyperglycemia. Moreover, the delayed gastric emptying promotes a heightened sense of satiety. In general, the heightened sense of satiety results in weight loss. Specifically, the neurotoxin is administered in doses ranging from 1,000 units/Kg to 30,000 units/Kg of body weight. In general, the toxin is produced by Clostridium Botulinum.

In yet another embodiment of the present invention, there is provided a method for treatment of post-prandial hyperglycemia in an individual comprising impairing gastric muscle contraction, thereby effectively treating post-prandial hyperglycemia in said individual. In one aspect of this embodiment, gastric muscle contraction is impaired by administering a neurotoxin in doses ranging from 1,000 units/Kg to 30,000 units/Kg of body weight. Preferably, the neurotoxin is made by a bacterium selected from the group consisting of Clostridium botulinum, Clostridium butyricum, and Clostridium barati. In one aspect, the neurotoxin is a botulinum toxin. Representative examples of botulinum toxin include but are not limited to botulinum toxin types A, B, C1, D, E, F and G. The neurotoxin may be administered into the antrum of the stomach, the fundus of the stomach or into both the antrum and the fundus of the stomach. In one aspect of this method, the individual has overt diabetes mellitus.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Clinical Trial

Study Design: Open label, pilot
Study Population: Morbidly obese patients (BMI ≧40) presenting to the Obesity Center.
Number of Patients: 20, at least 10 of whom will have overt diabetes (requiring insulin or oral antidiabetic agents)

### Exclusion Criteria

1. Age <18 or >65
2. BMI <40
3. History of frequent hypoglycemic episodes
4. HbA1C >10
5. Severe pulmonary or cardiovascular disease precluding conscious sedation for endoscopy
6. Known allergic reaction to botulinum toxin injection
7. Known delay in gastric emptying or symptoms suggestive of the same i.e. nausea and vomiting

### Pre-Injection Interventions

1. Standard counseling and nutritional advice to all patients for a 4 week period prior to BoNT injection
2. HbA1C one week before
3. Oral glucose tolerance test
4. Fasting and post-prandial levels of insulin, glucagon, ghrelin, amylin, GLP-1, GIP, NPY, PYY.
5. Satiety index using liquid test meal
6. Solid and liquid phase gastric emptying at baseline.

### Example 2

### Interventions:

### Upper Endoscopy and Injection of BoNT/B

Endoscopy is done using conscious sedation (typically a combination of midazolam and fentanyl). Botulinum neurotoxin is injected using an 8 mm sclerotherapy needle inserted via the working channel of the endoscope. Based on the animal studies, a good estimate of the optimal site and dose for use in humans will be possible. For purposes of the study, and based on experience, 10 mg of Botulinum neurotoxin in rats can be considered equivalent to about 100 units in humans.

### Post-Injection Measurements:

1. Daily weight and caloric intake measurement until end of study (2 consecutive weeks of weight gain)
2. Satiety index using liquid test meal at 2 weeks and then at 4; week intervals until end of study
3. Solid and liquid phase gastric emptying at baseline and at 4 weeks after injection of BoNT/B
4. Oral glucose tolerance test 2 weeks after injection and at 4 week intervals until end of study
5. Fasting and post-prandial levels of insulin, glucagon, ghrelin, amylin, GLP-1, GIP, NPY, PYY 2 weeks after injection and then at 4 week intervals until the end of study
6. HbA1C 12 weeks after

### Analysis:

1. Primary end point
   a. maximum weight loss after Botulinum neurotoxin
   b. Change in area under curve (AUC) in oral GTT after Botulinum neurotoxin
2. Secondary end points:
   a. Maximum decrease in caloric intake
   b. Duration of weight loss
   c. Change in satiety index
   d. Change in gastric emptying
   e. Change in hormonal levels
   f. Side-effects

### Example 3

### Sample Size and Power Calculations

This is an open label single cohort pilot study so formal sample size calculations have not been done. The report by Foschi et al randomized 24 patients to either placebo or botulinum neurotoxin. The group receiving Botulinum neurotoxin (n=12) demonstrated a reduction in weight of 11 kg with calculated standard deviation of about 3.5 kg. With respect to changes in postprandial hyperglycemia, in a single-blind, placebo-controlled, crossover study, 18 evaluable subjects with type 1 diabetes underwent two standardized breakfast meal tests and received pramlintide or placebo in addition to their preprandial insulin (Ceriello et al Diabetes Care 2005;28:632-37). Preprandial administration of pramlintide (which acts principally by retarding gastric emptying), as an adjunct to regular insulin, prevented the initial rise in postprandial plasma glucose and significantly reduced the overall glucose excursion observed with regular insulin alone (placebo). Mean incremental glucose AUC_{0-4 h} values (mean±SE) were −0.6±2.5 mmol·l⁻¹·h⁻¹ for pramlintide and +11.0±2.9 mmol·l⁻¹·h⁻¹ for placebo (P=0.001). Even if the effects of botulinum neurotoxin on gastric emptying are half as effective as pramlintide (botulinum neurotoxin injections can cause about a 25% decrease in gastric emptying according to the study by Foschi et al whereas pramlintide can cause a 50% reduction—Kong et al Diabetologia. May 1998;41(5):577-83), one could expect to detect a difference in this population of 20 patients.

### Example 4

### Risks Related to Upper Gastrointestinal Endoscopy

These are infrequent: infection (0.008%), perforation (0.03%), bleeding (0.03%) and reaction to sedatives (0.06%).

Risks Associated with Iniection Procedure Per Se

These are no significant risks expected from the process of injection itself as the needle is very short (8 mm) and thin (25G). Furthermore, patients who undergo pyloric injection of botulinum toxin for gastroparesis do not appear to have any serious complications as well. Thus, one should not expect any significant risks from injection of the toxin.

Risks related to the Toxin Itself

These appear to be mild and infrequent.

Risks Associated with Additional Radiological Tests.

The additional risks from participating in this study are related to the two additional radionuclide gastric emptying studies (one prior and one after injection of botulinum toxin or placebo into the pylorus). Radiation dose to the whole body from this exam is 0.0045 rad/250 microcuries which is less than the radiation dose from a chest X-ray. A small amount of radiation is also expected during the performance of antroduodenal manometery as fluoroscopy is used to guide the catheter into the duodenum.

### Example 5

### Procedures for Minimizing Risk

All endoscopies are performed only by experienced endoscopists. Conscious sedation is provided along standard guidelines with continuous monitoring of vital signs, cardiac rhythm and pulse oximetry. Adequate facilities are available for resuscitation. Radiological studies (gastric emptying studies) are preformed under standard precautions by experienced personnel.

### Example 6

### Monitoring of Serious Adverse Events

Serious adverse events defined as adverse events, are fatal or considered life-threatening, which require hospitalization or prolong hospitalization, cause permanent disability, constitute cancer, or overdose. These are reported to the IRB immediately and pending investigation by an independent committee, the trail will be suspended. During the first seven days after injection, patients are contacted daily and questioned for the occurrence of potential complications such as fever, abdominal pain, systemic weakness, flu-like illness. An adverse effect is defined as any symptom, physical sign, syndrome or disease, which occurs during the study, having been absent at baseline, or if present at baseline appears to worsen.

### Example 7

### Satiety Testing

A liquid caloric meal is given to test meal-induced satiety. In this test a peristaltic pump fills one of two beakers at a fixed rate of 15 mL/min with a liquid meal. The subjects are requested to maintain intake at the filling rate, thereby alternating the beakers as they were filled and emptied. At 5-minute intervals, they are asked to score their satiety using a graphic rating scale that combined verbal descriptors on a scale graded of 0-5 (1, threshold; 5, maximum satiety). Participants are instructed to stop meal intake when a score of 5 is reached.

### Example 8

### Gastric Emptying

This is performed according to the international consensus protocol by Tougas et al. Studies are started in the morning between 8 to 9 a.m. after an overnight fasting. The test meal is low-fat (egg-beaters) with a caloric value of 255 kcal and mixed with 1 mCi of Tc-99m sulfur colloid. Anterior and posterior images of the abdomen are taken within 1 minute after the completion of the meal (defined as time 0). Further images are obtained after 60, 120 and 240 minutes in all patients. With the patient sitting upright, 1 minute anterior and posterior images are taken using a single head camera in the 140 keV Tc-99m peak with a 20% window. Data are corrected for time decay. The region of interest is drawn around the image of the stomach for each time frame. For each time frame the geometric mean is calculated as the square root of the product of the counts measured on the anterior and posterior images. The main parameters measured are the percent gastric retention (normal values in parentheses) at 60 (<90%), 120 (<60%) and 240 minutes (<10%).

### Example 9

### Oral Glucose Tolerance Test and Hormonal Levels

The meal consists of a standardized breakfast, consisting of a bagel with margarine and jam, cheese, yogurt, milk, and orange juice. The size of the meal is calculated individually to provide 30% of a subject's total daily caloric requirements with a macronutrient composition according to the American Diabetes Association nutritional recommendations (55%/15%/30% of kcal from carbohydrate/protein/fat, respectively). The main pharmacodynamic parameters include the incremental plasma glucose area under the concentration time curve (AUC) from 0 to 2 h (AUC_{0-2 h}), incremental AUC_{0-4 h}, and the incremental plasma glucose concentrations at specific sampling times.

Any patents or publications mentioned in this specification are indicative of the level of those skilled in the art to which the invention pertains. Further, these patents and publications are incorporated by reference herein to the same extent as if each individual publication was specifically and individually incorporated by reference.

One skilled in the art would appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method for treatment of post-prandial hyperglycemia in an individual comprising:
administering a neurotoxin intragastrically to impair gastric muscle contraction, thereby effectively treating post-prandial hyperglycemia in said individual.

2. The method of claim 1, wherein said neurotoxin is administered in doses ranging from 1,000 units/Kg to 30,000 units/Kg of body weight.

3. The method of claim 1, wherein said neurotoxin is made by a bacterium selected from the group consisting of Clostridium botulinum, Clostridium butyricum, and Clostridium barati. toxin.

4. The method of claim 1, wherein the neurotoxin is a botulinum

5. The method of claim 4, wherein the botulinum toxin is selected from the group consisting of botulinum toxin types A, B, C1, D, E, F and G.

6. The method of claim 1, wherein the neurotoxin is administered into the antrum of the stomach.

7. The method of claim 1, wherein the neurotoxin is administered into the fundus of the stomach.

8. The method of claim 1, wherein the neurotoxin is administered into both the antrum and the fundus of the stomach.

9. The method of claim 1, wherein said impairment of the gastric muscle contraction results in a delayed emptying of the stomach.

10. The method of claim 1, wherein said impairment of the gastric muscle contraction results in altered neuropeptide release.

11. The method of claim 9, wherein said delay in emptying of the stomach promotes a sense of satiety.

12. The method of claim 10, wherein said neuropeptide is gherlin.

13. The method of claim 1, wherein said individual has overt Diabetes Mellitus.

14. A method for treatment of post-prandial hyperglycemia in an individual comprising:
administering a neurotoxin intragastrically to impair gastric muscle contraction, thereby effectively treating post-prandial hyperglycemia in said individual, wherein said neurotoxin is administered in doses ranging from 1,000 units/Kg to 30,000 units/Kg of body weight.

15. The method of claim 14, wherein said neurotoxin is made by a bacterium selected from the group consisting of Clostridium botulinum, Clostridium butyricum, and Clostridium barati.

16. The method of claim 14, wherein the neurotoxin is a botulinum toxin.

17. The method of claim 16, wherein the botulinum toxin is selected from the group consisting of botulinum toxin types A, B, C1, D, E, F and G.

18. The method of claim 14, wherein the neurotoxin is administered into the antrum of the stomach.

19. The method of claim 14, wherein the neurotoxin is administered into the fundus of the stomach.

20. The method of claim 14, wherein the neurotoxin is administered into both the antrum and the fundus of the stomach.

21. The method of claim 14, wherein said impairment of the gastric muscle contraction results in a delayed emptying of the stomach.

22. The method of claim 14, wherein said impairment of the gastric muscle contraction results in altered neuropeptide release.

23. The method of claim 14, wherein said individual has overt Diabetes Mellitus.

24. A method for treatment of post-prandial hyperglycemia in an individual comprising:
impairing gastric muscle contraction, thereby effectively treating post-prandial hyperglycemia in said individual.

25. The method of claim 24, wherein said gastric muscle contraction is impaired by administering a neurotoxin in doses ranging from 1,000 units/Kg to 30,000 units/Kg of body weight.

26. The method of claim 24, wherein said neurotoxin is made by a bacterium selected from the group consisting of Clostridium botulinum, Clostridium butyricum, and Clostridium barati.

27. The method of claim 24, wherein the neurotoxin is a botulinum toxin.

28. The method of claim 24, wherein the botulinum toxin is selected from the group consisting of botulinum toxin types A, B, C1, D, E, F and G.

29. The method of claim 24, wherein the neurotoxin is administered into the antrum of the stomach, the fundus of the stomach or into both the antrum and the fundus of the stomach.

30. The method of claim 24, wherein said individual has overt Diabetes Mellitus.
